(19) United States
(12) Patent Application Publication
(10) Pub. No.: US 2021/0337827 A1
(43) Pub. Date: Nov. 4, 2021

(54) GROWTH OF FILAMENTOUS FUNGI FROM PEA PROTEIN RESIDUAL WASTE STREAMS

(71) Applicant: EMERGY INC., Boulder, CO (US)

(72) Inventors: Justin WHITELEY, Lafayette, CO (US); Mitchell Tyler HUGGINS, Boulder, CO (US); Tracy YATES, Arvada, CO (US)

(73) Assignee: EMERGY INC., Boulder, CO (US)

(21) Appl. No.: 17/243,283

(22) Filed: Apr. 28, 2021

Related U.S. Application Data
(60) Provisional application No. 63/017,451, filed on Apr. 29, 2020.

(51) Int. Cl. A23J 3/22; A23J 3/14; A23L 31/00; C12N 1/14

(52) U.S. Cl. CPC A23J 3/227; A23J 3/14; A23V 2002/00; C12N 1/14; A23L 31/00

(57) ABSTRACT

A method of forming an edible meat substitute product includes obtaining a liquid waste stream from a pea protein processor. The method includes adjusting the pH of the liquid waste stream to be in a range of 5.5 to 6.0 so as to form a growth media. The method includes growing fungal cells in the growth media comprising the liquid waste stream obtained from a pea processor such that the fungal cells produce a mycelium mass. The method includes separating the mycelium mass from the growth media. The method includes concentrating the mycelium to obtain a fibrous mycelium mass having a protein content of greater than 40 wt % of a dry mass of the mycelium.

### CROSS-REFERENCE TO RELATED APPLICATIONS

The present applications claims priority to and benefit of U.S. Provisional Application No. 63/017,451, filed on Apr. 29, 2020, the entire disclosure of which is incorporated herein by reference.

### TECHNICAL FIELD

The present disclosure relates generally the field of fungal mycelium based edible meat substitute products.

### BACKGROUND

Demand for edible products that can provide a high protein content which is drawn from a non-animal source is increasing. Driven by increasing awareness of personal health, edible products that include non-animal sourced components such as proteins and fibers are considered as a healthier alternative to animal protein based products. In particular, there is growing demand for edible meat substitutes that mimic meat in its composition and texture, but are composed of non-animal components, which can reduce reliance on animals such as cows, chicken, and pigs, and reduce the carbon footprint posed by such animals. Thus, there is a need for non-animal protein sources that can facilitate large scale production and adoption of non-animal based edible products.

### SUMMARY

Embodiments described herein relate generally to methods for growing filamentous fungi from pea protein residual waste streams for obtaining edible meat substitute products that resemble animal meat in their texture and morphology.

In some embodiments, a method of forming an edible meat substitute product comprises obtaining a liquid waste stream from a pea protein processor; adjusting the pH of the liquid waste stream to be in a range of 5.5 to 6.0 so as to form a growth media; growing fungal cells in the growth media such that the fungal cells produce a mycelium mass; separating the mycelium mass from the growth media; and concentrating the mycelium mass to obtain a fibrous mycelium mass having a protein content of greater than 40 wt % of a dry mass of the mycelium.

In some embodiments, an edible meat substitute product is formed by the process of obtaining a liquid waste stream from a pea protein processor; adjusting the pH of the liquid waste stream to be in a range of 5.5 to 6.0 so as to form a growth media; growing fungal cells in the growth media such that the fungal cells produce a mycelium mass; separating the mycelium mass from the growth media; and concentrating the mycelium mass to obtain a fibrous mycelium mass having a protein content of greater than 40 wt % of a dry mass of the fibrous mycelium mass.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several implementations in accordance with the disclosure and are therefore, not to be considered limiting of its scope, the disclosure will be described with additional specificity and detail through use of the accompanying drawings.

FIG. 1 is a flow chart of an example method for growing filamentous fungi from pea protein residual waste streams, according to an embodiment.

FIG. 2 is a table listing the components of an example pea protein waste stream, according to an embodiment.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION

Embodiments described herein relate generally to methods for growing filamentous fungi from pea protein residual waste streams for obtaining edible meat substitute products that resemble animal meat in their texture and morphology. Particularly, various embodiments described herein provide methods of obtaining a liquid waste stream from a pea protein processor, adjusting the pH of the liquid waste stream to be in a range of 5.5 to 6.0 so as to form a growth media, growing fungal cells in the growth media such that the fungal cells produce a mycelium mass, separating the mycelium mass from the growth media, and concentrating the mycelium mass to obtain a fibrous mycelium mass having a protein content of greater than 40 wt % of a dry mass of the mycelium. Various embodiments also relate to adding food additives to form an edible food product or edible meat substitute product. The edible meat substitute product can include a mycelium mass having a protein content of greater than 40 wt % of a dry mass of the mycelium mass.

Various embodiments of the methods of growing fungal mycelium and forming edible products therefrom described herein may provide one or more benefits including, for example: (1) providing edible products that include protein from a non-animal source, i.e., fungal mycelium, thereby reducing dependence on animal sources of proteins and reducing their carbon footprint; (2) providing edible meat substitute products that feel and taste like real meat while delivering a high protein content; and (3) growing the edible meat substitute in recycled pea protein waste stream thereby reducing cost and waste.

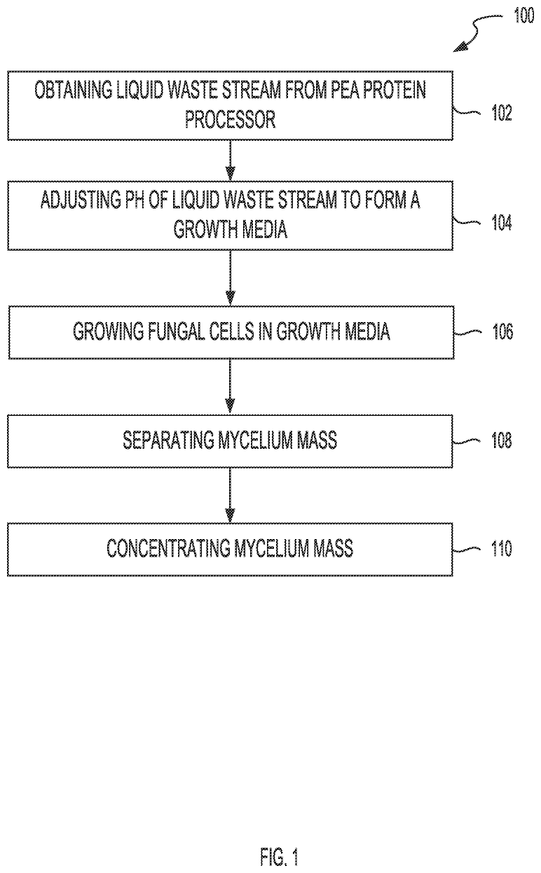

FIG. 1 illustrates a block diagram of an example method 100 for forming an edible meat substitute product, according to an embodiment. In brief overview, the method 100 may include obtaining a liquid waste stream from a pea protein processor, at 102. The method 100 may include adjusting the pH of the liquid waste stream to form a growth media, at 104. The method 100 may include growing fungal cells in a growth media, at 106. The method 100 may include separating mycelium mass from the growth media, at 108. The method 100 may include concentrating the mycelium mass, at 110.

In further detail, the method 100 may include obtaining a liquid waste stream from a pea protein processor, at 102. The liquid waste stream can include a protein content in a range of 10 wt % to 20 wt %. For example, the liquid waste stream can have a protein content of 10 wt %, 15 wt %, or 20 wt %, inclusive. The liquid waste stream can include a nitrogen content in a range of 1 wt % to 3 wt %. For example, the liquid waste stream can have a nitrogen content of 1 wt %, 1.5 wt %, 2.0 wt %, 2.5 wt %, or 3.0 wt %, inclusive. The liquid waste stream can include a carbohydrate content in a range of 15 wt % to 25 wt %. For example, the liquid waste stream can have a carbohydrate content of 15 wt %, 20 wt %, or 25 wt %, inclusive.

The liquid waste stream can include an ash content in a range of 5 wt % to 10 wt %. For example, the liquid waste stream can have an ash content of 5 wt %, 7 wt %, or 10 wt %, inclusive. The liquid waste stream can include a starch content in a range of 2 wt % to 4 wt %. For example, the liquid waste stream can have a starch content of 2 wt %, 2.5 wt %, 3 wt %, 3.5 wt %, or 4 wt %, inclusive. The liquid waste stream can include a potassium content in a range of 2 wt % to 4 wt %. For example, the liquid waste stream can have a potassium content of 2 wt %, 2.5 wt %, 3 wt %, 3.5 wt %, or 4 wt %, inclusive. The liquid waste stream can include a chloride content in a range of 2 wt % to 4 wt %. For example, the liquid waste stream can have a chloride content of 2 wt %, 2.5 wt %, 3 wt %, 3.5 wt %, or 4 wt %, inclusive. The liquid waste stream can include a fat content in a range of 0.5 wt % to 3 wt %. For example, the liquid waste stream can have a fat content of 0.5 wt %, 1 wt %, 1.5 wt %, 2.0 wt %, 2.5 wt %, or 3.0 wt %, inclusive. The liquid waste stream can include a crude fiber content of less than 0.5 wt %. For example, the liquid waste stream can have a crude fiber content of 0.4 wt %, 0.3 wt %, 0.2 wt %, 0.1 wt %, inclusive.

The liquid waste stream can include various amino acids. The liquid waste stream can include an alanine content in a range of 0.6 wt % to 0.7 wt %. For example, the liquid waste stream can have an alanine content of 0.6 wt %, 0.62 wt %, 0.64 wt %, 0.66 wt %, 0.68 wt %, or 0.7 wt %, inclusive. The liquid waste stream can include an arginine content in a range of 1.1 wt % to 1.3 wt %. For example, the liquid waste stream can have an arginine content of 1.1 wt %, 1.15 wt %, 1.2 wt %, 1.25 wt %, or 1.3 wt %, inclusive. The liquid waste stream can include an aspartic acid content in a range of 1.3 wt % to 1.5 wt %. For example, the liquid waste stream can have an aspartic acid content of 1.3 wt %, 1.35 wt %, 1.4 wt %, 1.45 wt %, or 1.5 wt %, inclusive. The liquid waste stream can include a cysteine content in a range of 0.2 wt % to 0.3 wt %. For example, the liquid waste stream can have a cysteine content of 0.2 wt %, 0.22 wt %, 0.24 wt %, 0.26 wt %, 0.28 wt %, or 0.3 wt %, inclusive. The liquid waste stream can include a glutamic acid content in a range of 2.6 wt % to 2.7 wt %. For example, the liquid waste stream can have a glutamic acid content of 2.6 wt %, 2.62 wt %, 2.64 wt %, 2.66 wt %, 2.68 wt %, or 2.7 wt %, inclusive.

The liquid waste stream can include a glycine content in a range of 0.7 wt % to 0.8 wt %. For example, the liquid waste stream can have a glycine content of 0.7 wt %, 0.72 wt %, 0.74 wt %, 0.76 wt %, 0.78 wt %, or 0.8 wt %, inclusive. The liquid waste stream can include a histidine content in a range of 0.3 wt % to 0.4 wt %. For example, the liquid waste stream can have a histidine content of 0.3 wt %, 0.32 wt %, 0.34 wt %, 0.36 wt %, 0.38 wt %, or 0.4 wt %, inclusive. The liquid waste stream can include an isoleucine content in a range of 0.3 wt % to 0.4 wt %. For example, the liquid waste stream can have an isoleucine content of 0.3 wt %, 0.32 wt %, 0.34 wt %, 0.36 wt %, 0.38 wt %, or 0.4 wt %, inclusive. The liquid waste stream can include a leucine content in a range of 0.4 wt % to 0.5 wt %. For example, the liquid waste stream can have a leucine content of 0.4 wt %, 0.42 wt %, 0.44 wt %, 0.46 wt %, 0.48 wt %, or 0.5 wt %, inclusive. The liquid waste stream can include a methionine content in a range of 0.01 wt % to 0.1 wt %. For example, the liquid waste stream can have a methionine content of 0.01 wt %, 0.02 wt %, 0.03 wt %, 0.04 wt %, 0.05 wt %, 0.06 wt %, 0.07 wt %, 0.08 wt %, 0.09 wt %, or 0.1 wt %, inclusive. The liquid waste stream can include a phenylalanine content in a range of 0.3 wt % to 0.4 wt %. For example, the liquid waste stream can have a phenylalanine content of 0.3 wt %, 0.32 wt %, 0.34 wt %, 0.36 wt %, 0.38 wt %, or 0.4 wt %, inclusive.

The liquid waste stream can include a proline content in a range of 0.4 wt % to 0.5 wt %. For example, the liquid waste stream can have a proline content of 0.4 wt %, 0.42 wt %, 0.44 wt %, 0.46 wt %, 0.48 wt %, or 0.5 wt %, inclusive. The liquid waste stream can include a serine content in a range of 0.5 wt % to 0.6 wt %. For example, the liquid waste stream can have a serine content of 0.5 wt %, 0.52 wt %, 0.54 wt %, 0.56 wt %, 0.58 wt %, or 0.6 wt %, inclusive. The liquid waste stream can include a threonine content in a range of 0.5 wt % to 0.6 wt %. For example, the liquid waste stream can have a threonine content of 0.5 wt %, 0.52 wt %, 0.54 wt %, 0.56 wt %, 0.58 wt %, or 0.6 wt %, inclusive. The liquid waste stream can include a total lysine content in a range of 0.5 wt % to 1.5 wt %. For example, the liquid waste stream can have a total lysine content of 0.5 wt %, 0.75 wt %, 1.0 wt %, 1.25 wt %, or 1.5 wt %, inclusive.

The liquid waste stream can include a tryptophan content in a range of 0.1 wt % to 0.2 wt %. For example, the liquid waste stream can have a tryptophan content of 0.1 wt %, 0.12 wt %, 0.14 wt %, 0.16 wt %, 0.18 wt %, or 0.2 wt %, inclusive. The liquid waste stream can include a tyrosine content in a range of 0.4 wt % to 0.5 wt %. For example, the liquid waste stream can have a tyrosine content of 0.4 wt %, 0.42 wt %, 0.44 wt %, 0.46 wt %, 0.48 wt %, or 0.5 wt %, inclusive. The liquid waste stream can include a valine content in a range of 0.4 wt % to 0.5 wt %. For example, the liquid waste stream can have a valine content of 0.4 wt %, 0.42 wt %, 0.44 wt %, 0.46 wt %, 0.48 wt %, or 0.5 wt %, inclusive.

The method 100 may include adjusting the pH of the liquid waste stream, at 104. For example, the pH of the liquid waste stream can be adjusted to a range of 5.5 to 6.0 (e.g., 5.5, 5.6, 5.7, 5.8, 5.9, or 6.0, inclusive). The pH of the liquid waste stream can be adjusted using 1M citric acid. For example, the pH of the liquid waste stream can be adjusted by adding citric acid to the liquid waste stream.

The method 100 may include growing fungal cells in a growth media, at 106. The fungal cells can include fungi from Ascomycota and Zygomycota, including the genera Aspergillus, Fusarium, Neurospora, and Monascus. Other species include edible varieties of Basidiomycota and genera Lentinula. One genus is Neurospora, which is used in food production through solid fermentation. The genus of Neurospora are known for highly efficient biomass production as well as ability to break down complex carbohydrates. For certain species of Neurospora, no known allergies have been detected and no levels of mycotoxins are produced. In addition to monocultures of filamentous fungi, multiple strains can be cultivated at once to tune the protein, amino acid, mineral, texture, and flavor profiles of the final biomass.

The growth media may be contained in a vessel, such as a vat capable of growing several kilograms of the fungal mycelium. The growth media can be referred to as an original growth media. The method 100 may include growing fungal cells in a growth media such that the fungal cells produce mycelium. In some embodiments, growing the fungal cells includes adding fungal cells into a bioreactor containing the growth media.

In some embodiments, growing the fungal cells includes maintaining the growth media at a temperature in a range of 30° C. to 35° C., a stirring rate in a range of 200 rpm to 300 rpm, and an airflow in a range of 0.1 vvm to 5 vvm (volume of air under standard conditions per volume of liquid per minute) for a time period. For example, growing the fungal cells can include maintaining the growth media at a temperature of 30° C., 31° C., 32° C., 33° C., 34° C., or 35° C., inclusive. Growing the fungal cells can include maintaining the growth media at a stirring rate of 200 rpm, 210 rpm, 220 rpm, 230 rpm, 240 rpm, 250 rpm, 260 rpm, 270 rpm, 280 rpm, 290 rpm, or 300 rpm, inclusive. Growing the fungal cells can include maintaining the growth media at an airflow of 0.1 vvm, 0.5 vvm, 1 vvm, 2 vvm, 3 vvm, 4 vvm, or 5 vvm, inclusive.

In some embodiments, the pH of the growth media is maintained in a range of 5.5 to 6.0 for the time period. For example, the pH of the growth media can be maintained at a pH of 5.5, 5.6, 5.7, 5.8, 5.9, or 6.0, inclusive, for a time period. The time period can include 1 hour, 5 hours, 10 hours, 15 hours, 20 hours, 24 hours, 36 hours, or 48 hours, inclusive.

In some embodiments, the method 100 may include removing a volume of a broth (e.g., siphoned broth). The siphoned broth can contain the fungal cells and the growth media. For example, the siphoned broth can include a solution containing the fungal cells and the growth media. Removing a volume of broth can include discretely removing a volume of broth. For example, a volume of broth can be siphoned from a container containing the broth in a batch process, or be continuously removed from the broth. For example, a volume of broth can flow out of the container containing the broth in a continuous process.

The method 100 may include adding fresh growth media to a container containing the broth. The broth can be a fermentation broth. Nutrients (e.g., sugar, phosphate-containing compound, or nitrogen-containing compound) can be added in a batch growth configuration. For example, the nutrients can be added after a predetermined amount of time (e.g., after 1 hour, 2 hours, 3 hours, 6 hours, or 12 hours, inclusive). The concentrations of none or at least one of the nutrients of the fresh growth media can be brought to the concentrations of nutrients of the original growth media described in operation 102. The fresh growth media can have a volume that is greater than, less than, or equal to a volume of growth media that was lost from the original growth media during growth of the fungal cells in the original growth media.

In one example, after 6 hours, the concentration of sugar, phosphate-containing compound, and nitrogen-containing compound in the fresh growth media is increased. Nutrients are added to the broth to create a new broth. Nutrients are added to the broth to bring the concentrations of sugar, phosphate-containing compound, and nitrogen-containing compound of the new broth to the concentrations of sugar, phosphate-containing compound, and nitrogen-containing compound, respectively of the original growth media.

In one example, after at least 12 hours, 50-95% of the broth can be removed. Fresh media can be added containing nutrients (e.g., sugar, phosphate-containing compound, or nitrogen-containing compound). The nutrient concentration of the broth can be increased by adding fresh growth media.

Nutrients can be added in a continuous growth configuration. For example, a volume of broth (e.g., 0.01 vol %, 1 vol %, 5 vol %, 10 vol %, 25 vol %, 50 vol %, or 95 vol %, inclusive) can be removed from the container containing the fungal cells and the growth media. Fresh growth media can be added to the container containing the broth. The fresh growth media can be provided as a continuous flow. The volume of the broth in the container can be monitored to stay at a specified level. For example, the volume of the broth in the container can stay at a fixed volume. The volume of fresh growth media that is added can be equal to the volume of broth that is lost from the container.

The method 100 includes separating the mycelium mass from the growth media, at 108. Separating the mycelium mass from the growth media can be performed using gravity straining, centrifugation, a belt press, a filter press, a mechanical press, a drum dryer, or any other suitable process. The separated mycelium mass can have a moisture content of greater than 90 wt %. For example, the separated mycelium mass can have a moisture content of 91 wt %, 92 wt %, 93 wt %, 94 wt %, 95 wt %, 96 wt %, 97 wt %, 98 wt %, or 99 wt %, inclusive. During the separation process, the mycelium mass can be washed with water, ethanol, acid, base or other solvent. Recovered filtrate can be reused or discarded. Cell walls of the mycelium mass can be disrupted, for example, through lysing. Lysis may be performed by adjusting the pH to below 4 or above 9, by adding lysis enzymes, by raising the temperature in a range of 40° C. and 60° C. in a range of 1 and 24 hours, or any other suitable lysis method. Following separation, additives (e.g., food additives) can be mixed with the mycelium mass. Additives can include vegetable or animal proteins, fats, emulsifiers, thickeners, stabilizers, and flavoring, for example, when the mycelium mass is being formed into an edible product.

The method 100 may include concentrating the mycelium mass, at 110. Concentrating the mycelium mass may include filtering the mycelium mass to obtain a fibrous mycelium mass. The fibrous mycelium mass can have a protein content of greater than 40 wt % of a dry mass of the mycelium. For example, the mycelium mass can have a protein content of 45 wt %, 50 wt %, 60 wt %, 70 wt %, 80 wt %, or 90 wt %, inclusive, of the dry mass of the mycelium mass.

In some embodiments, the method 100 may include autoclaving the growth media before growing the fungal cells in the growth media. For example, the growth media can exposed to temperatures and pressures that sterilizes the growth media. In some embodiments, the method 100 may include diluting the liquid waste stream by a factor in a range of 8 to 12. For example, the liquid waste stream can be diluted by a factor 8, 9, 10, 11, or 12, inclusive.

In some embodiments, the method 100 may include supplementing the diluted growth media with a second media. The second media can include NH₄Cl in a range of 2 g L⁻¹ to 3 g L⁻¹. For example, the second media can include 2.1 g L⁻¹ NH₄Cl, 2.2 g L⁻¹ NH₄Cl, 2.3 g L⁻¹ NH₄Cl, 2.4 g L⁻¹ NH₄Cl, 2.5 g L⁻¹ NH₄Cl, 2.6 g L⁻¹ NH₄Cl, 2.7 g L⁻¹ NH₄Cl, 2.8 g L⁻¹ NH₄Cl, 2.9 g L⁻¹ NH₄Cl, or 3.0 g L⁻¹ NH₄Cl, inclusive.

The second media can include KH₂PO₄ in a range of 1 g L⁻¹ to 3 g L⁻¹. For example, the second media can include 1.1 g L⁻¹ KH₂PO₄, 1.2 g L⁻¹ KH₂PO₄, 1.3 g L⁻¹ KH₂PO₄, 1.4 g L⁻¹ KH₂PO₄, 1.5 g L⁻¹ KH₂PO₄, 1.6 g L⁻¹ KH₂PO₄, 1.7 g L⁻¹ KH₂PO₄, 1.8 g L⁻¹ KH₂PO₄, 1.9 g L⁻¹ KH₂PO₄, 2.0 g L⁻¹ KH₂PO₄, 2.1 g L⁻¹ KH₂PO₄, 2.2 g L⁻¹ KH₂PO₄, 2.3 g L⁻¹ KH₂PO₄, 2.4 g L⁻¹ KH₂PO₄, 2.5 g L⁻¹ KH₂PO₄, 2.6 g L⁻¹ KH₂PO₄, 2.7 g L⁻¹ KH₂PO₄, 2.8 g L⁻¹ KH₂PO₄, 2.9 g L⁻¹ KH₂PO₄, or 3.0 g L⁻¹ KH₂PO₄, inclusive.

The second media can include Na₃C₆H₅O₇ in a range of 0.5 g L⁻¹ to 2 g L⁻¹. For example, the second media can include 0.5 g L⁻¹ Na₃C₆H₅O₇, 0.6 g L⁻¹ Na₃C₆H₅O₇, 0.7 g L⁻¹ Na₃C₆H₅O₇, 0.8 g L⁻¹ Na₃C₆H₅O₇, 0.9 g L⁻¹ Na₃C₆H₅O₇, 1.0 g L⁻¹ Na₃C₆H₅O₇, 1.1 g L⁻¹ Na₃C₆H₅O₇, 1.2 g L⁻¹ Na₃C₆H₅O₇, 1.3 g L⁻¹ Na₃C₆H₅O₇, 1.4 g L⁻¹ Na₃C₆H₅O₇, 1.5 g L⁻¹ Na₃C₆H₅O₇, 1.6 g L⁻¹ Na₃C₆H₅O₇, 1.7 g L⁻¹ Na₃C₆H₅O₇, 1.8 g L⁻¹ Na₃C₆H₅O₇, 1.9 g L⁻¹ Na₃C₆H₅O₇, or 2.0 g L⁻¹ Na₃C₆H₅O₇, inclusive.

The second media can include MgSO₄ in a range of 0.05 g L⁻¹ to 0.5 g L⁻¹. For example, the second media can include 0.05 g L⁻¹ MgSO₄, 0.1 g L⁻¹ MgSO₄, 0.15 g L⁻¹ MgSO₄, 0.2 g L⁻¹ MgSO₄, 0.25 g L⁻¹ MgSO₄, 0.3 g L⁻¹ MgSO₄, 0.35 g L⁻¹ MgSO₄, 0.4 g L⁻¹ MgSO₄, 0.45 g L⁻¹ MgSO₄, or 0.5 g L⁻¹ MgSO₄, inclusive.

The second media can include CaCl₂ in a range of 0.05 g L⁻¹ to 0.5 g L⁻¹. For example, the second media can include 0.05 g L⁻¹ CaCl₂, 0.1 g L⁻¹ CaCl₂, 0.15 g L⁻¹ CaCl₂, 0.2 g L⁻¹ CaCl₂, 0.25 g L⁻¹ CaCl₂, 0.3 g L⁻¹ CaCl₂, 0.35 g L⁻¹ CaCl₂, 0.4 g L⁻¹ CaCl₂, 0.45 g L⁻¹ CaCl₂, or 0.5 g L⁻¹ CaCl₂, inclusive.

The second media can include ZnSO₄ in a range of 1 g L⁻¹ to 10 g L⁻¹. For example, the second media can include 1 g L⁻¹ ZnSO₄, 1.5 g L⁻¹ ZnSO₄, 2 g L⁻¹ ZnSO₄, 2.5 g L⁻¹ ZnSO₄, 3 g L⁻¹ ZnSO₄, 3.5 g L⁻¹ ZnSO₄, 4 g L⁻¹ ZnSO₄, 4.5 g L⁻¹ ZnSO₄, 5 g L⁻¹ ZnSO₄, 5.5 g L⁻¹ ZnSO₄, 6 g L⁻¹ ZnSO₄, 6.5 g L⁻¹ ZnSO₄, 7 g L⁻¹ ZnSO₄, 7.5 g L⁻¹ ZnSO₄, 8 g L⁻¹ ZnSO₄, 8.5 g L⁻¹ ZnSO₄, 9 g L⁻¹ ZnSO₄, 9.5 g L⁻¹ ZnSO₄, or 10 g L⁻¹ ZnSO₄, inclusive.

The second media can include Fe(NH₄)₂(SO₄)₂ in a range of 0.5 g L⁻¹ to 2 g L⁻¹. For example, the second media can include 0.5 g L⁻¹ Fe(NH₄)₂(SO₄)₂, 0.6 g L⁻¹ Fe(NH₄)₂(SO₄)₂, 0.7 g L⁻¹ Fe(NH₄)₂(SO₄)₂, 0.8 g L⁻¹ Fe(NH₄)₂(SO₄)₂, 0.9 g L⁻¹ Fe(NH₄)₂(SO₄)₂, 1.0 g L⁻¹ Fe(NH₄)₂(SO₄)₂, 1.1 g L⁻¹ Fe(NH₄)₂(SO₄)₂, 1.2 g L⁻¹ Fe(NH₄)₂(SO₄)₂, 1.3 g L⁻¹ Fe(NH₄)₂(SO₄)₂, 1.4 g L⁻¹ Fe(NH₄)₂(SO₄)₂, 1.5 g L⁻¹ Fe(NH₄)₂(SO₄)₂, 1.6 g L⁻¹ Fe(NH₄)₂(SO₄)₂, 1.7 g L⁻¹ Fe(NH₄)₂(SO₄)₂, 1.8 g L⁻¹ Fe(NH₄)₂(SO₄)₂, 1.9 g L⁻¹ Fe(NH₄)₂(SO₄)₂, or 2.0 g L⁻¹ Fe(NH₄)₂(SO₄)₂, inclusive.

The second media can include CuSO₄ in a range of 0.05 g L⁻¹ to 0.5 g L⁻¹. For example, the second media can include 0.05 g L⁻¹ CuSO₄, 0.1 g L⁻¹ CuSO₄, 0.15 g L⁻¹ CuSO₄, 0.2 g L⁻¹ CuSO₄, 0.25 g L⁻¹ CuSO₄, 0.3 g L⁻¹ CuSO₄, 0.35 g L⁻¹ CuSO₄, 0.4 g L⁻¹ CuSO₄, 0.45 g L⁻¹ CuSO₄, or 0.5 g L⁻¹ CuSO₄, inclusive.

The second media can include MnSO₄ in a range of 0.01 g L⁻¹ to 0.5 g L⁻¹. For example, the second media can include 0.01 g L⁻¹ MnSO₄, 0.02 g L⁻¹ MnSO₄, 0.03 g L⁻¹ MnSO₄, 0.04 g L⁻¹ MnSO₄, 0.05 g L⁻¹ MnSO₄, 0.1 g L⁻¹ MnSO₄, 0.15 g L⁻¹ MnSO₄, 0.2 g L⁻¹ MnSO₄, 0.25 g L⁻¹ MnSO₄, 0.3 g L⁻¹ MnSO₄, 0.35 g L⁻¹ MnSO₄, 0.4 g L⁻¹ MnSO₄, 0.45 g L⁻¹ MnSO₄, or 0.5 g L⁻¹ MnSO₄, inclusive.

The second media can include BH₃O₃ in a range of 0.01 g L⁻¹ to 0.5 g L⁻¹. For example, the second media can include 0.01 g L⁻¹ BH₃O₃, 0.02 g L⁻¹ BH₃O₃, 0.03 g L⁻¹ BH₃O₃, 0.04 g L⁻¹ BH₃O₃, 0.05 g L⁻¹ BH₃O₃, 0.1 g L⁻¹ BH₃O₃, 0.15 g L⁻¹ BH₃O₃, 0.2 g L⁻¹ BH₃O₃, 0.25 g L⁻¹ BH₃O₃, 0.3 g L⁻¹ BH₃O₃, 0.35 g L⁻¹ BH₃O₃, 0.4 g L⁻¹ BH₃O₃, 0.45 g L⁻¹ BH₃O₃, or 0.5 g L⁻¹ BH₃O₃, inclusive.

The second media can include Na₂MoO₄ in a range of 0.01 g L⁻¹ to 0.5 g L⁻¹. For example, the second media can include 0.01 g L⁻¹ Na₂MoO₄, 0.02 g L⁻¹ Na₂MoO₄, 0.03 g L⁻¹ Na₂MoO₄, 0.04 g L⁻¹ Na₂MoO₄, 0.05 g L⁻¹ Na₂MoO₄, 0.1 g L⁻¹ Na₂MoO₄, 0.15 g L⁻¹ Na₂MoO₄, 0.2 g L⁻¹ Na₂MoO₄, 0.25 g L⁻¹ Na₂MoO₄, 0.3 g L⁻¹ Na₂MoO₄, 0.35 g L⁻¹ Na₂MoO₄, 0.4 g L⁻¹ Na₂MoO₄, 0.45 g L⁻¹ Na₂MoO₄, or 0.5 g L⁻¹ Na₂MoO₄, inclusive.

The second media can include biotin in a range of 0.01 g L⁻¹ to 0.5 g L⁻¹. For example, the second media can include 0.01 g L⁻¹ biotin, 0.02 g L⁻¹ biotin, 0.03 g L⁻¹ biotin, 0.04 g L⁻¹ biotin, 0.05 g L⁻¹ biotin, 0.1 g L⁻¹ biotin, 0.15 g L⁻¹ biotin, 0.2 g L⁻¹ biotin, 0.25 g L⁻¹ biotin, 0.3 g L⁻¹ biotin, 0.35 g L⁻¹ biotin, 0.4 g L⁻¹ biotin, 0.45 g L⁻¹ biotin, or 0.5 g L⁻¹ biotin, inclusive.

In some embodiments, the method 100 may include forming the fibrous mycelium mass into an edible food product. For example, the edible food product can include a chip, a protein bar, a jerky, a tortilla, a bread, or a cracker. The edible food product can include a chicken substitute product, a beef substitute product, a veal substitute product, or a fish substitute product.

Following are some examples of growing filamentous fungi from pea protein residual waste streams. These examples are for illustrative purposes only and should not be construed as limiting the disclosure in any shape or form.

In one example, a liquid waste stream from a pea protein processor is obtained. The waste stream was added to a 3 L benchtop bioreactor at 2 L working volume and autoclaved. Following autoclaving, pH was adjusted to 5.8 using 1M citric acid. 50 mL of Neurospora crassa was added as a seed to the bioreactor. The bioreactor was operated for 24 h at 32° C., with an agitation of 250 rpm, airflow of 1 vvm, and a constant pH of 5.8. At the end of the run, biomass was filtered from the solution and measured. The dry weight was approximately 12 g L⁻¹. This run represents the ability to use this discarded pea protein processing waste stream as a filamentous fungi nutrient media. In some embodiments, the waste stream is certified organic allowing for access to an organic sugar source. In some embodiments, the flavor of the waste stream is minimal and thus the final biomass had minimal flavor.

The fibrous mycelium mass can be used in a single or combination of ways. For example, the fibrous mycelium mass can be cooked at a temperature of less than 100° C. (e.g., 90° C., 80° C., 75° C., or 50° C., inclusive) for 1-60 minutes in dry or steam environment. The fibrous mycelium mass can be cooked at a temperature range of 100° C. to 200° C. (e.g., 100° C., 125° C., 150° C., or 200° C., inclusive) for 1-60 minutes in dry or steam environment. The fibrous mycelium mass can be cooked in a water bath at less than 100° C. for 1 minute to 120 minutes (e.g., 1, 2, 5, 10, 20, 40, 60, 80, 100, or 120 minutes, inclusive).

In some embodiments, the fibrous mycelium mass can be stored. The fibrous mycelium mass can include additional ingredients. The fibrous mycelium mass can be cooked. The fibrous mycelium mass can be frozen at less than 0° C. under ambient or vacuum conditions, and/or refrigerated at less than 5° C. under ambient or vacuum conditions. The fibrous mycelium mass can be stored indefinitely in sealed container.

Producing the fibrous mycelium mass can include tuning the texture of the fibrous mycelium mass. Texture of the fibrous mycelium mass can be tuned by chemical washing of the mycelium mass. Alternatively, texture can be altered by controlling the water content of the mycelium mass. Texture can also be altered through the addition of different nutrients which determine mycelium mass growth and morphology. The density of final mycelium mass can be controlled by altering initial water content and drying conditions to produce a heavier or lighter end product.

The edible meat substitute product can be formed by process described above by method 100. The edible meat substitute product can be formed by the process of obtaining a liquid waste stream from a pea protein processor. The edible meat substitute product can be formed by the process of adjusting the pH of the liquid waste stream to be in a range of 5.5 to 6.0 so as to form a growth media. The edible meat substitute product can be formed by the process of growing fungal cells in the growth media comprising the liquid waste stream obtained from a pea processor such that the fungal cells produce a mycelium mass. The edible meat substitute product can be formed by the process of separating the mycelium mass from the growth media. The edible meat substitute product can be formed by the process of concentrating the mycelium to obtain a fibrous mycelium mass having a protein content of greater than 40 wt % of a dry mass of the mycelium.

The edible meat substitute product can include a fibrous mycelium mass in a range of 10 wt % to 100 wt % (e.g., 10 wt %, 20 wt %, 30 wt %, 40 wt %, 50 wt %, 60 wt %, 70 wt %, 80 wt %, or 100 wt %, inclusive). The edible meat substitute product can have a water content in a range of 0 wt % to 100 wt % (e.g., 0 wt %, 10 wt %, 20 wt %, 30 wt %, 40 wt %, 50 wt %, 60 wt %, 70 wt %, 80 wt %, or 100 wt %, inclusive). In some embodiments, the fibrous mycelium mass is in a range of 10 wt % to 50 wt %, and the water content is in a range of 50 wt % to 90 wt %. In some embodiments, the edible meat substitute product includes a soluble protein in a range of 1 wt % to 20 wt % (e.g., 1 wt %, 2 wt %, 5 wt %, 10 wt %, 15 wt %, or 20 wt %, inclusive). The edible meat substitute product can include a thickener content in a range of 0.01 wt to 5 wt % (e.g., 0.01 wt %, 0.05 wt %, 0.1 wt %, 1 wt %, 2 wt %, or 5 wt %, inclusive). The edible meat substitute product can include a fat source in a range of 0 wt % to 10 wt % (e.g., 0 wt %, 0.5 wt %, 1 wt %, 2 wt %, 5 wt %, or 10 wt %, inclusive).

The edible meat substitute product can include a flavorant. A flavorant can include flavorings or food additives. For example, the flavorant can include an oil, such as a nut-derived oil, vegetable-derived oil, plant-derived oil, and animal-derived oil. The flavorant can include spices (e.g., black pepper, fennel, mustard, nutmeg, cinnamon, ginger, cayenne pepper, clove, etc.). The flavorant can include a flavored powder (e.g., onion powder, garlic powder, BBQ powder, sour cream powder, lemon powder, lime powder, etc.).

The edible meat substitute product can include a combined methionine and cysteine content of at least 20 mg/gram crude protein. In some embodiments, the combined methionine and cysteine content in the edible meat substitute product is in a range of 20 mg/gram to 30 mg/gram (e.g., 20 mg/gram, 25 mg/gram, or 30 mg/gram, inclusive). The edible meat substitute product can have a PDCAAS score of 1. The edible meat substitute product can have an internal pH in a range of 2 to 9 (e.g., 2, 3, 4, 5, 6, 7, 8, or 9, inclusive). The edible meat substitute product can have a protein dry weight in a range of 20 wt % to 70 wt % (e.g., 20 wt %, 30 wt %, 40 wt %, 50 wt %, 60 wt %, or 70 wt %, inclusive). The edible meat substitute product can have a fiber dry weight in a range of 5 wt % to 30 wt % (e.g., 5 wt %, 10 wt %, 15 wt %, 20 wt %, 25 wt %, or 30 wt %, inclusive). The edible meat substitute product can have a dry fat weight of 0 wt % to 20 wt % (e.g., 0 wt %, 1 wt %, 5 wt %, 10 wt %, 15 wt %, or 20 wt %, inclusive). The edible meat substitute product can have a color represented by a CIE L* value of greater than 55. The edible meat substitute product can have a Warner-Bratzler shear force of greater than 15 N. The edible meat substitute product can have a hardness of greater than 0.003 kgf/mm² (e.g., in a range of 0.0035 kgf/mm² to 0.018 kgf/mm², inclusive).

The edible meat substitute product can include a chicken substitute product, a beef substitute product, a pork substitute product, a veal substitute product, or a fish substitute product. The edible meat substitute product can include 10 wt % to 90 wt % of the fibrous mycelium mass (e.g., 10 wt %, 20 wt %, 30 wt %, 40 wt %, 50 wt %, 60 wt %, 70 wt %, 80 wt %, or 90 wt %, inclusive).

The chicken substitute product can include 50-90 wt % water (e.g., 50 wt %, 60 wt %, 70 wt %, 80 wt %, or 90 wt %, inclusive). The chicken substitute product can include 10-50 wt % fungal mycelium such as from N. crassa (e.g., 10 wt %, 20 wt %, 30 wt %, 40 wt %, or 50 wt %, inclusive). The chicken substitute product can include 1-20 wt % soluble protein (e.g., 1 wt %, 2 wt %, 5 wt %, 10 wt %, or 20 wt %, inclusive). The soluble protein can include pea, egg white, and potato, among others. The chicken substitute product can include 0.01-5 wt % thickener (e.g., 0.01 wt %, 0.05 wt %, 0.1 wt %, 1 wt %, 2 wt %, or 5 wt %, inclusive). The thickener can include pectin, carrageenan, agar, among others. The chicken substitute product can include 0-10 wt % fat source (0 wt %, 1 wt %, 2 wt %, 3 wt %, 4 wt %, 5 wt %, or 10 wt %, inclusive). The fat source can include vegetable oils, seeds, among others. The chicken substitute product can include seasonings. The chicken substitute product can have various physical properties. For example, the chicken substitute product can have an internal pH in a range of 2 and 9 (e.g., 2, 3, 4, 5, 6, 7, 8, or 9, inclusive). The chicken substitute product can have a 40-70 wt % protein dry weight (e.g., 40 wt %, 45 wt %, 50 wt %, 55 wt %, 60 wt %, 65 wt %, or 70 wt %, inclusive). The chicken substitute product can have a 5-30 wt % fiber dry weight (e.g., 5 wt %, 10 wt %, 15 wt %, 20 wt %, 25 wt %, or 30 wt %, inclusive). The chicken substitute product can have a 0-10 wt % fat dry weight (0 wt %, 1 wt %, 2 wt %, 4 wt %, 5 wt %, or 10 wt %, inclusive). The chicken substitute product can have a CIE L* value greater than 55. The chicken substitute product can have a Warner-Bratzler shear force greater than 15 N. The chicken substitute product can have a hardness greater than 0.003 kgf/mm² (e.g., in a range of 0.003 kgf/mm² to 0.018 kgf/mm², inclusive).

The meat substitute product can include 0-90 wt % water (e.g., 0 wt %, 10 wt %, 20 wt %, 30 wt %, 40 wt %, 50 wt %, 60 wt %, 70 wt %, 80 wt %, or 90 wt %, inclusive). The meat substitute product can include 10-100 wt % fungal mycelium such as from N. crassa (e.g., 10 wt %, 20 wt %, 30 wt %, 40 wt %, 50 wt %, 60 wt %, 70 wt %, 80 wt %, 90 wt %, or 100 wt %, inclusive). The meat substitute product can include 1-20 wt % soluble protein (e.g., 1 wt %, 2 wt %, 5 wt %, 10 wt %, or 20 wt %, inclusive). The soluble protein can include pea, egg white, and potato, among others. The meat substitute product can include 0-5 wt % thickener (e.g., 0 wt %, 0.01 wt %, 0.05 wt %, 0.1 wt %, 1 wt %, 2 wt %, or 5 wt %, inclusive). The thickener can include pectin, carrageenan, agar, among others. The meat substitute product can include 0-50 wt % fat source (0 wt %, 10 wt %, 20 wt %, 30 wt %, 40 wt %, or 50 wt %, inclusive). The fat source can include vegetable oils, seeds, among others. The meat substitute product can include seasonings.

The fibrous mycelium mass flavor can be enhanced by adding different oils. Non-limiting examples of oils include nut-derived, vegetable-derived, plant-derived, and animal-derived oils. Oils can be added to the food-grade residual water streams to have the multi-purpose of acting as an antifoaming agent, a carbon source for the fungus, and to integrate extra/intracellularly into the mycelium mass. Alternatively, oil can be integrated into the mycelium mass following harvesting or following cooking.

Texture of the fibrous mycelium mass can be tuned by chemical washing of the fibrous mycelium mass. Alternatively, texture can be altered by controlling the water content of the fibrous mycelium mass. Texture can also altered through the addition of different nutrients which determine fibrous mycelium mass growth and morphology. The density of final fibrous mycelium mass can be controlled by altering initial water content and drying conditions to produce a heavier or lighter end product.

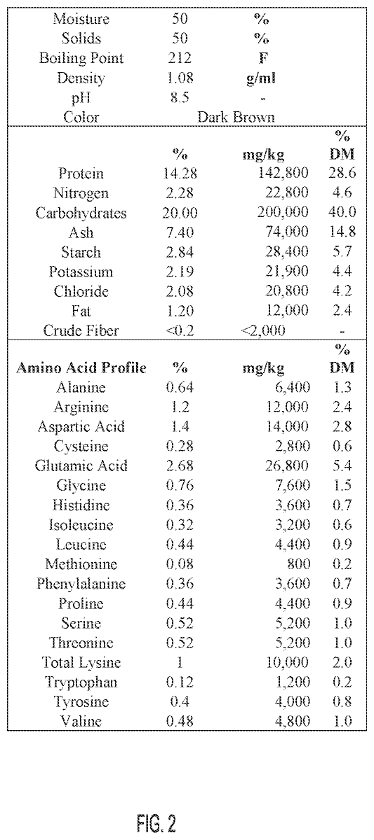

FIG. 2 is a table listing constituents of an example pea protein waste stream. The pea protein waste stream can have a moisture content of about 50 wt %. The pea protein waste stream can have a solid content of about 50 wt %. The pea protein waste stream can have a boiling point of about 100° C. (212° F.). The pea protein waste stream can have a density of about 1.08 g/mL. The pea protein waste stream can have a pH of about 8.5. The pea protein waste stream can have a dark brown color.

The pea protein waste stream can have a protein content of about 14.28 wt %. The pea protein waste stream can have a protein content of about 142,800 mg/kg. The pea protein waste stream can have a protein content of about 28.6 DM % (dry matter %). The pea protein waste stream can have a nitrogen content of about 2.28 wt %. The pea protein waste stream can have a nitrogen content of about 22,800 mg/kg. The pea protein waste stream can have a nitrogen content of about 4.6 DM %. The pea protein waste stream can have a carbohydrate content of about 20.00 wt %. The pea protein waste stream can have a carbohydrate content of about 200,000 mg/kg. The pea protein waste stream can have a carbohydrate content of about 40.0 DM %. The pea protein waste stream can have an ash content of about 7.40 wt %. The pea protein waste stream can have an ash content of about 74,000 mg/kg. The pea protein waste stream can have an ash content of about 14.8 DM %. The pea protein waste stream can have a starch content of about 2.84 wt %. The pea protein waste stream can have a starch content of about 28,400 mg/kg. The pea protein waste stream can have a starch content of about 5.7 DM %.

The pea protein waste stream can have a potassium content of about 2.19 wt %. The pea protein waste stream can have a potassium content of about 21,900 mg/kg. The pea protein waste stream can have a potassium content of about 4.4 DM %. The pea protein waste stream can have a chloride content of about 2.08 wt %. The pea protein waste stream can have a chloride content of about 20,800 mg/kg. The pea protein waste stream can have a chloride content of about 4.2 DM %. The pea protein waste stream can have a fat content of about 1.20 wt %. The pea protein waste stream can have a fat content of about 12,000 mg/kg. The pea protein waste stream can have a fat content of about 2.4 DM %. The pea protein waste stream can have a crude fiber content of less than about 0.2 wt %. The pea protein waste stream can have a fat content of less than about 2,000 mg/kg.

The pea protein waste stream can include various amino acids. The pea protein waste stream can include an alanine content in a range of 0.6 wt % to 0.7 wt %. For example, the pea protein waste stream can have an alanine content of 0.6 wt %, 0.62 wt %, 0.64 wt %, 0.66 wt %, 0.68 wt %, or 0.7 wt %, inclusive. The pea protein waste stream can include an arginine content in a range of 1.1 wt % to 1.3 wt %. For example, the pea protein waste stream can have an arginine content of 1.1 wt %, 1.15 wt %, 1.2 wt %, 1.25 wt %, or 1.3 wt %, inclusive. The pea protein waste stream can include an aspartic acid content in a range of 1.3 wt % to 1.5 wt %. For example, the pea protein waste stream can have an aspartic acid content of 1.3 wt %, 1.35 wt %, 1.4 wt %, 1.45 wt %, or 1.5 wt %, inclusive. The pea protein waste stream can include a cysteine content in a range of 0.2 wt % to 0.3 wt %. For example, the pea protein waste stream can have a cysteine content of 0.2 wt %, 0.22 wt %, 0.24 wt %, 0.26 wt %, 0.28 wt %, or 0.3 wt %, inclusive. The pea protein waste stream can include a glutamic acid content in a range of 2.6 wt % to 2.7 wt %. For example, the pea protein waste stream can have a glutamic acid content of 2.6 wt %, 2.62 wt %, 2.64 wt %, 2.66 wt %, 2.68 wt %, or 2.7 wt %, inclusive.

The pea protein waste stream can include a glycine content in a range of 0.7 wt % to 0.8 wt %. For example, the pea protein waste stream can have a glycine content of 0.7 wt %, 0.72 wt %, 0.74 wt %, 0.76 wt %, 0.78 wt %, or 0.8 wt %, inclusive. The pea protein waste stream can include a histidine content in a range of 0.3 wt % to 0.4 wt %. For example, the pea protein waste stream can have a histidine content of 0.3 wt %, 0.32 wt %, 0.34 wt %, 0.36 wt %, 0.38 wt %, or 0.4 wt %, inclusive. The pea protein waste stream can include an isoleucine content in a range of 0.3 wt % to 0.4 wt %. For example, the pea protein waste stream can have an isoleucine content of 0.3 wt %, 0.32 wt %, 0.34 wt %, 0.36 wt %, 0.38 wt %, or 0.4 wt %, inclusive. The pea protein waste stream can include a leucine content in a range of 0.4 wt % to 0.5 wt %. For example, the pea protein waste stream can have a leucine content of 0.4 wt %, 0.42 wt %, 0.44 wt %, 0.46 wt %, 0.48 wt %, or 0.5 wt %, inclusive. The pea protein waste stream can include a methionine content in a range of 0.01 wt % to 0.1 wt %. For example, the pea protein waste stream can have a methionine content of 0.01 wt %, 0.02 wt %, 0.03 wt %, 0.04 wt %, 0.05 wt %, 0.06 wt %, 0.07 wt %, 0.08 wt %, 0.09 wt %, or 0.1 wt %, inclusive. The pea protein waste stream can include a phenylalanine content in a range of 0.3 wt % to 0.4 wt %. For example, the pea protein waste stream can have a phenylalanine content of 0.3 wt %, 0.32 wt %, 0.34 wt %, 0.36 wt %, 0.38 wt %, or 0.4 wt %, inclusive.

The pea protein waste stream can include a proline content in a range of 0.4 wt % to 0.5 wt %. For example, the pea protein waste stream can have a proline content of 0.4 wt %, 0.42 wt %, 0.44 wt %, 0.46 wt %, 0.48 wt %, or 0.5 wt %, inclusive. The pea protein waste stream can include a serine content in a range of 0.5 wt % to 0.6 wt %. For example, the pea protein waste stream can have a serine content of 0.5 wt %, 0.52 wt %, 0.54 wt %, 0.56 wt %, 0.58 wt %, or 0.6 wt %, inclusive. The pea protein waste stream can include a threonine content in a range of 0.5 wt % to 0.6 wt %. For example, the pea protein waste stream can have a threonine content of 0.5 wt %, 0.52 wt %, 0.54 wt %, 0.56 wt %, 0.58 wt %, or 0.6 wt %, inclusive. The pea protein waste stream can include a total lysine content in a range of 0.5 wt % to 1.5 wt %. For example, the pea protein waste stream can have a total lysine content of 0.5 wt %, 0.75 wt %, 1.0 wt %, 1.25 wt %, or 1.5 wt %, inclusive. The pea protein waste stream can include a tryptophan content in a range of 0.1 wt % to 0.2 wt %. For example, the pea protein waste stream can have a tryptophan content of 0.1 wt %, 0.12 wt %, 0.14 wt %, 0.16 wt %, 0.18 wt %, or 0.2 wt %, inclusive. The pea protein waste stream can include a tyrosine content in a range of 0.4 wt % to 0.5 wt %. For example, the pea protein waste stream can have a tyrosine content of 0.4 wt %, 0.42 wt %, 0.44 wt %, 0.46 wt %, 0.48 wt %, or 0.5 wt %, inclusive. The pea protein waste stream can include a valine content in a range of 0.4 wt % to 0.5 wt %. For example, the pea protein waste stream can have a valine content of 0.4 wt %, 0.42 wt %, 0.44 wt %, 0.46 wt %, 0.48 wt %, or 0.5 wt %, inclusive.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

As used herein, the singular forms “a”, “an”, and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings and tables in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated in a single software product or packaged into multiple software products.

Thus, particular implementations of the invention have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method, comprising:
obtaining a liquid waste stream from a pea protein processor;
adjusting the pH of the liquid waste stream to be in a range of 5.5 to 6.0 so as to form a growth media;
growing fungal cells in the growth media such that the fungal cells produce a mycelium mass;
separating the mycelium mass from the growth media; and
concentrating the mycelium mass to obtain a fibrous mycelium mass having a protein content of greater than 40 wt % of a dry mass of the mycelium.

2. The method of claim 1, wherein the pH is adjusted by adding citric acid to the liquid waste stream.

3. The method of claim 1, wherein the liquid waste stream comprises 10 wt % to 20 wt % protein, 1 wt % to 3 wt % nitrogen, 15 wt % to 25 wt % carbohydrate, 5 wt % to 10 wt % ash, 2 wt % to 4 wt % starch, 2 wt % to 4 wt % potassium, 2 wt % to 4 wt % chloride, 0.5 wt % to 3 wt % fat, and less than 0.5 wt % crude fiber.

4. The method of claim 1, further comprising:
autoclaving the growth media before growing the fungal cells in the growth media.

5. The method of claim 4, wherein growing the fungal cells comprises:
adding fungal cells into a bioreactor containing the growth media; and
maintaining the growth media at a temperature in a range of 30° C. to 35° C., a stirring rate in a range of 200 rpm to 300 rpm, and an airflow in a range of 0.1 vvm to 5 vvm for a time period,
wherein the pH of the growth media is maintained in a range of 5.5 and 6.0 for the time period.

6. The method of claim 1, further comprising:
diluting the liquid waste stream by a factor in a range of 8 to 12.

7. The method of claim 6, further comprising:
supplementing the diluted growth media with a second media, the second media comprising:
2.67 g L⁻¹ NH₄Cl;
2 g L⁻¹ KH₂PO₄;
1 g L⁻¹ Na₃C₆H₅O₇;
0.2 g L⁻¹ MgSO₄;
0.1 g L⁻¹ CaCl₂;
5 mg L⁻¹ ZnSO₄;
1 mg L⁻¹ Fe(NH₄)₂(SO₄)₂;
0.25 mg L⁻¹ CuSO₄;
0.05 mg L⁻¹ MnSO₄;
0.05 mg L⁻¹ BH₃O₃;
0.05 mg L⁻¹ Na₂MoO₄; and
0.05 mg L⁻¹ biotin.

8. The method of claim 1, further comprising:
forming the fibrous mycelium mass into an edible food product.

9. The method of claim 8, wherein the edible food product is at least one of a chip, a protein bar, a jerky, a tortilla, a bread, or a cracker.

10. The method of claim 8, wherein the edible food product is at least one of a chicken substitute product, a beef substitute product, a veal substitute product, or a fish substitute product.

11. An edible meat substitute product formed by the process of:
obtaining a liquid waste stream from a pea protein processor;
adjusting the pH of the liquid waste stream to be in a range of 5.5 to 6.0 so as to form a growth media;
growing fungal cells in the growth media such that the fungal cells produce a mycelium mass;
separating the mycelium mass from the growth media; and
concentrating the mycelium mass to obtain a fibrous mycelium mass having a protein content of greater than 40 wt % of a dry mass of the fibrous mycelium mass.

12. The edible meat substitute product formed by the process of claim 11, wherein the pH is adjusted by adding citric acid to the liquid waste stream.

13. The edible meat substitute product formed by the process of claim 11, wherein the liquid waste stream comprises 10 wt % to 20 wt % protein, 1 wt % to 3 wt % nitrogen, 15 wt % to 25 wt % carbohydrate, 5 wt % to 10 wt % ash, 2 wt % to 4 wt % starch, 2 wt % to 4 wt % potassium, 2 wt % to 4 wt % chloride, 0.5 wt % to 3 wt % fat, and less than 0.5 wt % crude fiber.

14. The edible meat substitute product formed by the process of claim 11, further comprising:
autoclaving the growth media before growing the fungal cells in the growth media.

15. The edible meat substitute product formed by the process of claim 11, wherein growing the fungal cells comprises:
adding fungal cells into a bioreactor containing the growth media; and
maintaining the growth media at a temperature in a range of 30° C. to 35° C., a stirring rate in a range of 200 rpm to 300 rpm, and an airflow in a range of 0.1 vvm to 5 vvm for a time period, and
wherein the pH of the growth media is maintained in a range of 5.5 and 6.0 for the time period.

16. The edible meat substitute product formed by the process of claim 11, further comprising:
diluting the liquid waste stream by a factor in a range of 8 to 12.

17. The edible meat substitute product formed by the process of claim 16, further comprising:
supplementing the diluted growth media with a second media, the second media comprising:
2.67 g L⁻¹ NH₄Cl;
2 g L⁻¹ KH₂PO₄;
1 g L⁻¹ Na₃C₆H₅O₇;
0.2 g L⁻¹ MgSO₄;
0.1 g L⁻¹ CaCl₂;
5 mg L⁻¹ ZnSO₄;
1 mg L⁻¹ Fe(NH₄)₂(SO₄)₂;
0.25 mg L⁻¹ CuSO₄;
0.05 mg L⁻¹ MnSO₄;
0.05 mg L⁻¹ BH₃O₃;
0.05 mg L⁻¹ Na₂MoO₄; and
0.05 mg L⁻¹ biotin.

18. The edible meat substitute product formed by the process of claim 11, wherein the edible meat substitute product is a chicken substitute product.

19. The edible meat substitute product formed by the process of claim 11, wherein the edible meat substitute product is a beef substitute product.

20. The edible meat substitute product formed by the process of claim 11, wherein the edible meat substitute product is a fish substitute product.
